(19) United States
(12) Patent Application Publication
(10) Pub. No.: US 2024/0374720 A1
(43) Pub. Date: Nov. 14, 2024

(54) ANTIBODY FORMULATIONS AND USES THEREOF

(71) Applicant: Amgen Inc., Thousand Oaks, CA (US)

(72) Inventors: Anna IP, Moorpark, CA (US); Ketaki PATEL, Simi Valley, CA (US); Clea TALLEY, Camarillo, CA (US); Michael J. TREUHEIT, Newbury Park, CA (US); Jun ZHANG, Newbury Park, CA (US)

(21) Appl. No.: 17/928,561

(22) PCT Filed: May 28, 2021
(86) PCT No.: PCT/US2021/034987

§ 371 Date: Nov. 29, 2022

Related U.S. Application Data
(60) Provisional application No. 63/031,634, filed on May 29, 2020.

(51) Int. Cl. A61K 39/395; A61K 47/12; A61K 47/18; A61K 47/26; C07K 16/18

(52) U.S. Cl. CPC A61K 39/39591; A61K 47/12; A61K 47/183; A61K 47/26; C07K 16/18

(57) ABSTRACT

Antibody formulations and methods of making and using such formulations are provided herein. The formulation can be for intravenous administration. In some embodiments, the formulation is for subcutaneous administration. In some embodiments, the formulation comprises an anti-C5 antibody, such as eculizumab.

### RELATED APPLICATIONS

This application is a U.S. national stage filing under 35 U.S.C. § 371 of PCT Application No. PCT/US21/34987, filed on May 28, 2021, which claims the benefit of U.S. Provisional Application No. 63/031,634, filed on May 29, 2020, both of which are hereby incorporated by reference in its entirety.

### SEQUENCE LISTING

The present application is being filed along with a Sequence Listing in electronic format. The Sequence Listing is provided as a file entitled A-2590-US02-PCT-Final-SeqListing.txt, created May 1, 2023, which is 21.3 KB in size. The information in the electronic format of the Sequence Listing is incorporated herein by reference in its entirety.

### FIELD OF THE INVENTION

The instant disclosure relates to formulations for an antibody and methods for making and using such formulations.

### BACKGROUND

The complement system comprises a series of proteins that interact with one another in a cascade fashion as part of an immune response, serving a complementary role alongside the antibody immune response, having an important role in host defense against microorganisms and in the modulation of inflammatory reactions. Complement is activated by three pathways: the classical pathway, alternative pathway and lectin pathway, in which each pathway initially involves different proteins, but all pathways converge with the cleavage of complement component C3. C3 is cleaved into C3a, which promotes inflammation and recruit circulating immune cells, while C3b forms a complex with other components to initiate a cascade of reactions among the later components of the complement system. C3b complexes with other complement components to form the C5-convertase complex. Complement component C5 is cleaved by the C5-convertase complex into C5a and C5b. C5a promotes inflammation, such as by acting as a chemoattractant for inflammatory cells. C5b remains attached to the cell surface where it triggers the formation of the membrane attack complex (MAC). The MAC is a hydrophilic pore that spans the membrane and promotes the free flow of fluid into and out of the cell, thereby destroying it.

Complement system dysregulation can result in different pathological conditions. Cells express proteins that protect them from the effects of the complement cascade to ensure that targets of the complement system are limited to pathogenic cells. Many complement-related disorders and diseases are associated with abnormal destruction of self cells by the complement cascade. An example of such disorder is paroxysmal nocturnal hemoglobinuria (PNH), PNH can arise from a genetic mutation that depletes one or more cytoprotective proteins that prevent destruction of red blood cells platelets and other blood cells from complement-mediated attack and can be characterized by hemolytic anemia (a decreased number of RBCs due to cell lysis), hemoglobinuria (hemoglobin in the urine due to RBC lysis), and/or hemoglobinemia (free hemoglobin in the bloodstream due to RBC lysis).

A therapeutic that can be used to treat a complement-related disorder is an agent that can inhibit C5 cleavage, such as an antibody that binds complement C5. One example of such a therapeutic is eculizumab, which is marketed as SolirisR (Alexion Pharmaceuticals, Inc., New Haven, CT). Another example is ravulizumab, which is marketed as UltomirisR (Alexion Pharmaceuticals, Inc., New Haven, CT).

The present disclosure provides formulations that meet the need for antibody formulations that are stable, have less aggregation, and/or other advantages.

### SUMMARY

Provided herein are antibody formulations and methods for making and using such formulations. The formulation can be a pharmaceutical formulation or pharmaceutical composition. In some embodiments, the antibody is an anti-C5 antibody.

In one embodiment, the formulation comprises a buffer, a stabilizer and a chelating agent. In some embodiments, the buffer comprises acetate. The concentration of acetate can be from 0.1 mM to 50 mM, such as from 0.5 mM to 50 mM, from 1 mM to 50 mM, from 2.5 mM to 40 Mm, from 5 mM to 30 mM, or from 10 mM to 20 mM. In one embodiment, the acetate concentration is about 10 mM. In some embodiments, the stabilizer of the formulation is a polyol, such as sorbitol. In one embodiment, the concentration of the polyol, such as sorbitol, is about 5% (w/v). In one embodiment, the concentration of the chelating agent in the formulation is between 0.01 mM and 0.05 mM, such as about 0.05 mM. In one embodiment, the chelating agent is ethylenediaminetetraacetic acid (EDTA). In some embodiments, the concentration of the surfactant is between 0.001% and 0.1% (w/v), such as 0.01% (w/v). In some embodiments, the surfactant is polysorbate 80. The formulation can have a pH within the buffering capacity of acetate. In one embodiment, the pH is between 4.5 and 5.8. In on embodiment, the pH is about 5.2.

In some embodiments, the antibody is eculizumab or ravulizumab. In some embodiments, the antibody can comprise CDRH1-3, wherein CDRH1-3 has the amino acid sequence of SEQ ID NOs: 1-3 or 4, 5, and 3, respectively. In one embodiment, the antibody comprises CDRL1-3, wherein the amino acid sequence of CDRL1-3 is SEQ ID NOs: 12-14, respectively. In some embodiments, the antibody comprises a heavy chain variable region of SEQ ID NO: 6 or 7. In some embodiments, the antibody comprises a light chain variable region of SEQ ID NO: 15. In some embodiments, the antibody comprises a heavy chain constant region having the amino acid sequence of SEQ ID NO: 8 or 9. In some embodiments, the antibody comprises a light chain constant region having the amino acid sequence of SEQ ID NO: 16. In some embodiments, the antibody comprises a heavy chain having the amino acid sequence of SEQ ID NO: 10 or 11. In some embodiments, the antibody comprises a light chain having the amino acid sequence of SEQ ID NO: 17. In another embodiment, the antibody comprises a heavy chain comprising the amino acid sequence of SEQ ID NO: 10 and a light chain comprising the amino acid sequence of SEQ ID NO: 17. In another embodiment, the antibody comprises a heavy chain comprising the amino acid sequence of SEQ ID NO: 11 and a light chain comprising the amino acid sequence of SEQ ID NO: 17. In some embodiments, the concentration of the antibody is about 10 mg/ml.

### BRIEF DESCRIPTION OF THE DRAWINGS

FIG. 1 shows the percentage of high molecular weight species as measured by SE-UHPLC at 25° C. for 10 mg/ml of eculizumab in a sorbitol formulation with no detectable trace metals, a sorbitol formulation with detectable trace metals (eculizumab from two different lots, Lot A and Lot B), and a PBS formulation, for 6 months.

FIG. 2 shows the relative potency (%) of eculizumab in a PBS formulation, sorbitol formulation without EDTA, and sorbitol formulation with EDTA at 50° C.

FIG. 3 shows the percentage of oxidation forms of eculizumab as measured by HIC-HPLC pre-peaks in sorbitol formulations with varying levels of EDTA at 40° C. for 13 weeks.

FIG. 4 shows the percentage of high molecular weight species as measured by SE-UHPLC in sorbitol formulations with varying levels of EDTA at 40° C. for 13 weeks.

FIG. 5 shows the percentage of high molecular weight species as measured by SE-UHPLC for eculizumab drug product (DP) produced using non-SUS (GMP DP1, GMP DP2, GMP DP3) and eculizumab DP produced using SUS (SUS DP) under forced degradation conditions (FD) of incubation at 50° C. for 14 days.

FIG. 6A shows the percentage of oxidation of W107 of eculizumab drug product (DP) produced using non-SUS (GMP 1 DP, GMP 2 DP) and eculizumab DP produced using SUS (SUS DP) under forced degradation conditions (FD) of incubation at 50° C. for 2 weeks.

FIG. 6B shows the relative potency (%) of eculizumab drug product (DP) produced using non-SUS (GMP 1 DP, GMP 2 DP) and eculizumab DP produced using SUS (SUS DP) under forced degradation conditions (FD) of incubation at 50°° C. for 2 weeks.

FIG. 7 shows the percentage of Main Peak as measured by HIC-HPLC of eculizumab drug product (DP) produced using non-SUS (GMP 2 DP) and eculizumab DP produced using SUS (SUS DP) under forced degradation conditions (FD) of incubation at 50° C. for 2 weeks.

FIG. 8 shows the percentage of Pre-Peaks as measured by HIC-HPLC of eculizumab drug product (DP) produced using non-SUS (GMP 2 DP) and eculizumab DP produced using SUS (SUS DP) under forced degradation conditions (FD) of incubation at 50° C. for 2 weeks.

### DETAILED DESCRIPTION

The instant disclosure provides antibody formulations and methods for making and using such formulations. In one embodiment, the antibody is an antibody that specifically binds to the complement protein C5. The antibody can be an anti-C5 antibody. In one embodiment, the antibody is eculizumab. In another embodiment, the antibody is ravulizumab.

In one embodiment, the antibody comprises heavy chain CDRs having the amino acid sequence of GYIFSNYWIQ (SEQ ID NO: 1) for CDRH1, the amino acid sequence of EILPGSGSTEYTENFKD (SEQ ID NO: 2) for CDRH2, and the amino acid sequence of YFFGSSPNWYFDV (SEQ ID NO: 3) for CDRH3. In some embodiments, the antibody comprises heavy chain CDRs having the amino acid sequence of the amino acid sequence of GHIFSNYWIQ (SEQ ID NO: 4) for CDRH1, the amino acid sequence of EILPGSGHTEYTENFKD (SEQ ID NO: 5) for CDRH2, and the amino acid sequence of SEQ ID NO: 3 for CDRH3.

In one embodiment, the antibody comprises a heavy chain variable domain having an amino acid sequence of:

(SEQ ID NO: 6)
QVQLVQSGAEVKKPGASVKVSCKASGYIFSNYWIQWVRQAPGQGL
EWMGEILPGSGSTEYTENFKDRVTMTRDTSTSTVYMELSSLRSED
TAVYYCARYFFGSSPNWYFDVWGQGTLVTVSS

In one embodiment, the antibody comprises a heavy chain variable region having an amino acid sequence of:

(SEQ ID NO: 7)
QVQLVQSGAEVKKPGASVKVSCKASGHIFSNYWIQWVRQAPGQGL
EWMGEILPGSGHTEYTENFKDRVTMTRDTSTSTVYMELSSLRSED
TAVYYCARYFFGSSPNWYFDVWGQGTLVTVSS

In one embodiment, the antibody comprises a heavy chain constant region having an amino acid sequence of:

(SEQ ID NO: 8)
ASTKGPSVFPLAPCSRSTSESTAALGCLVKDYFPEPVTVSWNSGA
LTSGVHTFPAVLQSSGLYSLSSVVTVPSSNFGTQTYTCNVDHKPS
NTKVDKTVERKCCVECPPCPAPPVAGPSVFLFPPKPKDTLMISRT
PEVTCVVVDVSQEDPEVQFNWYVDGVEVHNAKTKPREEQFNSTYR
VVSVLTVLHQDWLNGKEYKCKVSNKGLPSSIEKTISKAKGQPREP
QVYTLPPSQEEMTKNQVSLTCLVKGFYPSDIAVEWESNGQPENNY
KTTPPVLDSDGSFFLYSRLTVDKSRWQEGNVFSCSVMHEALHNHY
TQKSLSLSLGK

In one embodiment, the antibody comprises a heavy chain constant region having an amino acid sequence of:

(SEQ ID NO: 9)
ASTKGPSVFPLAPCSRSTSESTAALGCLVKDYFPEPVTVSWNSGA
LTSGVHTFPAVLQSSGLYSLSSVVTVPSSNFGTQTYTCNVDHKPS
NTKVDKTVERKCCVECPPCPAPPVAGPSVFLFPPKPKDTLMISRT
PEVTCVVVDVSQEDPEVQFNWYVDGVEVHNAKTKPREEQFNSTYR
VVSVLTVLHQDWLNGKEYKCKVSNKGLPSSIEKTISKAKGQPREP
QVYTLPPSQEEMTKNQVSLTCLVKGFYPSDIAVEWESNGQPENNY
KTTPPVLDSDGSFFLYSRLTVDKSRWQEGNVFSCSVLHEALHSHY
TQKSLSLSLGK

In one embodiment, the antibody comprises a heavy chain having an amino acid sequence of:

(SEQ ID NO: 10)
QVQLVQSGAEVKKPGASVKVSCKASGYIFSNYWIQWVRQAPGQGL
EWMGEILPGSGSTEYTENFKDRVTMTRDTSTSTVYMELSSLRSED
TAVYYCARYFFGSSPNWYFDVWGQGTLVTVSSASTKGPSVFPLAP
CSRSTSESTAALGCLVKDYFPEPVTVSWNSGALTSGVHTFPAVLQ
SSGLYSLSSVVTVPSSNFGTQTYTCNVDHKPSNTKVDKTVERKCC
VECPPCPAPPVAGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVMHEALHNHYTQKSLSLSLGK

In one embodiment, the antibody comprises a heavy chain having an amino acid sequence of:

(SEQ ID NO: 11)
QVQLVQSGAEVKKPGASVKVSCKASGHIFSNYWIQWVRQAPGQGL
EWMGEILPGSGHTEYTENFKDRVTMTRDTSTSTVYMELSSLRSED
TAVYYCARYFFGSSPNWYFDVWGQGTLVTVSSASTKGPSVFPLAP
CSRSTSESTAALGCLVKDYFPEPVTVSWNSGALTSGVHTFPAVLQ
SSGLYSLSSVVTVPSSNFGTQTYTCNVDHKPSNTKVDKTVERKCC
VECPPCPAPPVAGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVLHEALHSHYTQKSLSLSLGK

In some embodiments, the antibody comprises light chain CDRs having the amino acid sequence of GASENIYGALN (SEQ ID NO: 12) for CDRL1, the amino acid sequence of GATNLAD (SEQ ID NO: 13) for CDRL2, and the amino acid sequence of QNVLNTPLT (SEQ ID NO: 14) for CDRL2.

In one embodiment, the antibody comprises a light chain variable region having an amino acid sequence of:

(SEQ ID NO: 15)
DIQMTQSPSSLSASVGDRVTITCGASENIYGALNWYQQKPGKAPK
LLIYGATNLADGVPSRFSGSGSGTDFTLTISSLQPEDFATYYCQN
VLNTPLTFGQGTKVEIK

In one embodiment, the antibody comprises a light chain constant region having an amino acid sequence of:

(SEQ ID NO: 16)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC

In one embodiment, the antibody comprises a light chain having an amino acid sequence of:

(SEQ ID NO: 17)
DIQMTQSPSSLSASVGDRVTITCGASENIYGALNWYQQKPGKAPK
LLIYGATNLADGVPSRFSGSGSGTDFTLTISSLQPEDFATYYCQN
VLNTPLTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

In one embodiment, the antibody comprises a heavy chain comprising CDRH1-3, wherein CDRH1-3 comprises the amino acid sequences of SEQ ID NOs: 1-3, respectively: and a light chain comprising CDRL1-3, wherein CDRL1-3 comprises the amino acid sequences of SEQ ID NOs: 12-14, respectively. In another embodiment, the antibody comprises a heavy chain comprising CDRH1-3, wherein CDRH1-3 comprises the amino acid sequences of SEQ ID NOs: 4, 5, and 3, respectively: and a light chain comprising CDRL1-3, wherein CDRL1-3 comprises the amino acid sequences of SEQ ID NOs: 12-14, respectively.

In another embodiment, the antibody comprises a heavy chain comprising a variable region comprising the amino acid sequence of SEQ ID NO: 6; and a light chain comprising a variable region comprising the amino acid sequence of SEQ ID NO: 15. In another embodiment, the antibody comprises a heavy chain comprising a variable region comprising the amino acid sequence of SEQ ID NO: 7; and a light chain comprising a variable region comprising the amino acid sequence of SEQ ID NO: 15.

In yet another embodiment, the antibody comprises a heavy chain comprising a variable region and a constant region comprising the amino acid sequences of SEQ ID NOs: 6 and 8, respectively; and a light chain comprising a variable region and a constant region comprising the amino acid sequences of SEQ ID NOs: 15 and 16, respectively. In yet another embodiment, the antibody comprises a heavy chain comprising a variable region and a constant region comprising the amino acid sequences of SEQ ID NOs: 7 and 9, respectively; and a light chain comprising a variable region and a constant region comprising the amino acid sequences of SEQ ID NOs: 15 and 16, respectively.

In another embodiment, the antibody comprises a heavy chain comprising the amino acid sequence of SEQ ID NO: 10 and a light chain comprising the amino acid sequence of SEQ ID NO: 17. In another embodiment, the antibody comprises a heavy chain comprising the amino acid sequence of SEQ ID NO: 11 and a light chain comprising the amino acid sequence of SEQ ID NO: 17.

In some embodiments, between 1 and 300 mg/ml of the anti-C5 antibody is present in a formulation disclosed herein. In some embodiments, the formulations described herein comprises between 1 and 50 mg/ml, between 1 and 300 mg/ml, between 1 and 250 mg/ml, between 1 and 200 mg/ml, between 1 and 100 mg/ml of the anti-C5 antibody. In one embodiment, the formulation comprises between 10 and 50 mg/ml of the anti-C5 antibody. In one embodiment, the formulation comprises less than 300 mg/ml, less than 250 mg/ml, less than 200 mg/ml, less than 100 mg/ml, less than 50 mg/ml, less than 45 mg/ml, less than 40 mg/ml, less than 30 mg/ml, or less than 25 mg/ml of the anti-C5 antibody. In one embodiment, the formulation comprises about 300 mg/ml, about 250 mg/ml, about 200 mg/ml, about 100 mg/ml, about 50 mg/ml, about 45 mg m/l, about 40 mg/ml, about 30 mg/ml, about 25 mg/ml, about 10 mg/ml, or about 5 mg/ml of the anti-C5 antibody. In one embodiment, the formulation comprises about 10 mg/ml of the anti-C5 antibody.

In some embodiments, the formulation comprises an anti-C5 antibody (e.g., about 10 mg/ml of an antibody comprising a heavy chain having the amino acid sequence of SEQ ID NO: 10 and a light chain sequence of SEQ ID NO: 17), a buffering agent (e.g., a buffer comprising 10 mM of acetate), a chelating agent (e.g., 0.05 mM EDTA) and optionally, a stabilizer (e.g., 5% sorbitol (w/v)) and/or a surfactant (e.g., 0.01% polysorbate 80). In some embodiments, the pH of the formulation is about 5.2

In some embodiments, the formulation comprises a buffering agent, such as acetate. In one embodiment, the concentration of the acetate or acetate buffer is from 0.1 mM to 50 mM, from 0.5 mM to 50 mM, between 1 mM to 50 mM, from 1 mM to 40 mM, from 2.5 mM to 40 mM, from 1 mM to 30 mM, from 1 mM to 20 mM, from 1 mM to 10 mM, from 1 mM to 5 mM, from 5 mM to 30 mM, or from 10 mM to 20 mM. In one embodiment, the concentration of the acetate or acetate buffer is about 0.5 mM, about 1 mM, about 2.5 mM, about 5 mM, about 10 mM, about 20 mM, about 25 mM, about 30 mM, about 40 mM, or about 50 mM. In one embodiment, the concentration of the acetate or acetate buffer is 10 mM.

In some embodiments, the formulation has a pH that is between 4.5 and 5.8. In some embodiments, the formulation has a pH that is about 4.5, 4.6, 4.7, 4.8, 4.9, 5.0, 5.1, 5.2, 5.3, 5.4, 5.5, 5.6, 5.7, or 5.8. In one embodiment, the pH of the formulation is about 5.2.

In some embodiments, the formulation comprises a stabilizer. In one embodiment, the stabilizer is a polyol or sugar. In some embodiments, the stabilizer is sucrose, sorbitol, glycerol, trehalose (e.g., α, α-trehalose or trehalose dihydrate), mannitol, dextrose, dextran, glucose, or any combination thereof. In one embodiment, the stabilizer is sorbitol. The concentration of the stabilizer can be between 0 and 50% (w/v) of the stabilizer. In some embodiments, the formulation comprises between 0 and 25% (w/v) of the stabilizer. In some embodiments, the formulation comprises between 0 and 20% (w/v), between 5 and 50% (w/v), between 10 and 20% (w/v), between 0 and 10% (w/v), between 5 and 10% (w/v) or between 2 and 10% (w/v) of a stabilizer. In some embodiments, the formulation comprises about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% (w/v) of a stabilizer, such as sorbitol. In one embodiment, the formulation comprises about 5% (w/v) of sorbitol.

In one embodiment, the formulation comprises a chelating agent. The chelating agent can be 1,4,7,10-tetraazacyclododecane-1,4,7,10-tetraacetic acid (DOTA), 1,4,7-triazacyclononane, 1-glutaric acid-4,7 acetic acid (NODAGA), 1,4,7-triazacyclononane-1,4,7-triacetic acid (NOTA), hydrazine-nicotinic acid (HYNIC), mercaptoacetylglycyltriglycine (MAG3), ethylenediaminetetraacetic acid (EDTA), triethylenetetramine (TETA), iminodiacetic acid, diethylenetriamine-N,N,N′,N′,N″-pentaacetic acid (DTPA) and/or combinations thereof. In one embodiment, the chelating agent is EDTA. The concentration of the chelating agent can be between 0.01 mM to 0.10 mM, such as about 0.01 mM, 0.02 mM, 0.03 mM, 0.04 mM, 0.05 mM, 0.06 mM, 0.07 mM, 0.08 mM, 0.09 mM, or 0.10 mM. In one embodiment, the concentration of the chelating agent, such as EDTA, is about 0.05 mM.

In one embodiment, the formulation also comprises a surfactant. The surfactant can be a polyoxyethylene glycol alkyl ether, a polyoxypropylene glycol alkyl ether, a glucoside alkyl ether, a polyoxyethylene glycol octylphenol ether, a polyoxyethylene glycol alkylphenol ether, a glycerol alkyl ester, a polyoxyethylene glycol sorbitan alkyl ester, a sorbitan alkyl ester, a cocamide MEA, a cocamide DEA, a dodecyldimethylamine oxide, a poloxamer, a polyethoxylated tallow amine (POEA), or a combination thereof. In one embodiment, the surfactant is a polysorbate. In one embodiment, the surfactant is polysorbate 20. In another embodiment, the surfactant is polysorbate 80. In yet another embodiment, the surfactant is a poloxamer, such as poloxamer 188. In one embodiment, the surfactant is Pluronic® F-68. In some embodiments, the formulation comprises from 0.001 to 3% (w/v), 0.001 to 2% (w/v), 0.001 to 1% (w/v), 0.001 to 0.5% (w/v) or 0.01% to 0.1% (w/v) of a surfactant. In some embodiments, the formulation comprises about 0.01% (w/v) of a surfactant, such as polysorbate 80.

In some embodiments, the formulation further comprises one or more additional excipient(s) or agent(s), such as a preservative, buffer, tonicity agent, antioxidant, stabilizer, nonionic wetting or clarifying agent, and/or viscosity-increasing agent.

In some embodiments, the formulations disclosed herein are used for intravenous injection or infusion (IV), subcutaneous injection (SC), intraperitoneal (IP) injection, intraocular injection, intraarticular injection, or intramuscular injection (IM).

The formulation can be used for treating or preventing a complement-associated disorder, such as rheumatoid arthritis (RA); antiphospholipid antibody syndrome; lupus nephritis; ischemia-reperfusion injury; atypical hemolytic uremic syndrome (aHUS); typical or infectious hemolytic uremic syndrome (tHUS); dense deposit disease (DDD); paroxysmal nocturnal hemoglobinuria (PNH); neuromyelitis optica (NMO) or neuromyelitis optica spectrum disorder (NMOSD); multifocal motor neuropathy (MMN); multiple sclerosis (MS); macular degeneration (e.g., age-related macular degeneration (AMD)); hemolysis, elevated liver enzymes, and low platelets (HELLP) syndrome; thrombotic thrombocytopenia purpura (TTP); spontaneous fetal loss; Pauci-immune vasculitis; epidermolysis bullosa; recurrent fetal loss; or traumatic brain injury. In some embodiments, the complement-associated disorder is a complement-associated vascular disorder such as a diabetes-associated vascular disorder, central retinal vein occlusion, a cardiovascular disorder, myocarditis, a cerebrovascular disorder, a peripheral vascular disorder, a renovascular disorder, a mesenteric/enteric vascular disorder, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy, or percutaneous transluminal coronary angioplasty (PTCA). In some embodiments, the complement-associated disorder is myasthenia gravis (MG), cold agglutinin disease, dermatomyositis, Graves' disease, atherosclerosis, Alzheimer's disease, Guillain-Barre Syndrome, Degos' disease, graft rejection (e.g., transplant rejection), sepsis, burn (e.g., severe burn), systemic inflammatory response sepsis, septic shock, spinal cord injury, glomerulonephritis, Hashimoto's thyroiditis, type I diabetes, psoriasis, pemphigus, autoimmune hemolytic anemia (AIHA), idiopathic thrombocytopenia purpura (ITP), Goodpasture syndrome, antiphospholipid syndrome (APS), or catastrophic APS (CAPS). In some embodiments, the formulation described herein can be used in methods for treating thrombotic microangiopathy (TMA), such as TMA associated with a complement-associated disorder. In one embodiment, the formulation is for treating PNH, aHUS, generalized MG (gMG) or refractory gMG, and/or NMOSD.

In some embodiments, the formulation is a concentrated solution of an anti-C5 antibody that can be diluted into a pharmaceutically-acceptable diluent for, e.g., systemic delivery of the antibody to the subject. In one embodiment, the formulation is in a single unit vial of 300 mg of the anti-C5 antibody at a concentration of 10 mg/mL and can be diluted to a final concentration of 5 mg/mL. The diluent can be a sodium chloride solution (e.g., 0.45% or 0.9% sodium chloride), dextrose solution (e.g., 5% dextrose in water), or Ringer's solution.

In some embodiments, a formulation comprising an anti-C5 antibody with a chelating agent (e.g., EDTA) has greater stability than a formulation without a chelating agent. For example, a formulation comprising an anti-C5 antibody (e.g., 10 mg/ml of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) can be more stable than the same formulation without EDTA (i.e., 10 mg/ml of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)).

In one embodiment, a formulation comprising an anti-C5 antibody with a chelating agent (e.g., EDTA) has greater stability than a formulation without a chelating agent after a given time period (e.g., about 1 day, about 2 days, about 3 days, about 4 days, about 5 days, about 6 days, about 7 days, about 8 days, about 9 days, about 10 days, about 11 days, about 12 days, about 13 days, about 14 days or about 15 days: or about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks). In one embodiment, a formulation comprising an anti-C5 antibody with a chelating agent (e.g., EDTA) has greater stability than a formulation without a chelating agent at a given temperature or stress condition, such as at about 50° C., 40° C., about 30° C., about 25° C., about 5° C., about −20° C. or about −30° C.

In one embodiment, a formulation comprising an anti-C5 antibody with a chelating agent (e.g., EDTA) has greater stability than a formulation without a chelating agent after a given time period and a given temperature (e.g., about 1 day, about 2 days, about 3 days, about 4 days, about 5 days, about 6 days, about 7 days, about 8 days, about 9 days, about 10 days, about 11 days, about 12 days, about 13 days, about 14 days or about 15 days; or about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks; at about 50° C., 40° C., about 30° C., about 25° C., about 5° C., about −20° C. or about −30° C.).

The stability of a formulation can be determined by any method known in the art. In one embodiment, stability of a formulation is determined by chromatography, such as size exclusion chromatography, e.g., size exclusion high performance liquid chromatography (SE-HPLC) or size exclusion ultra high performance liquid chromatography (SE-UHPLC), or hydrophobic high performance liquid chromatography (HIC-HPLC), in which a lower change or difference in a first peak from a first formulation before a stress process and/or storage condition as compared to a second peak from the same formulation after the stress process and/or storage condition as compared to a second formulation with a greater change or difference in its first and second peaks before and after a stress process and/or storage condition, respectively, indicates the first formulation is more stable than the second formation.

In another embodiment, stability of a formulation is determined by the turbidity of the formulation (e.g., such as measured at OD₄₀₅ nm), percent of protein recovered (e.g., determined by SE-HPLC), and/or purity of protein (e.g., determined by SE-HPLC), in which lower turbidity, higher percentage of recovery and higher purity indicates higher stability. In some embodiments, SDS-PAGE (reducing or non-reducing) is used to determine the stability of a formulation. In some embodiments, CE-SDS (reducing or non-reducing) is used to determine the stability of a formulation. In some embodiments, asymmetric flow field-flow fractionation (AF4) is used. In other embodiments, isoelectric focusing (IEF), e.g., capillary isoelectric focusing (cIEF), is used. In some embodiments, AEX-HPLC is used. Increased fragments and/or changes in IEF in a first formulation as compared to a second formulation would indicate the first formulation is less stable. Any one method or combination of methods can be used to determine the stability of a formulation.

In some embodiments, a formulation comprising an anti-C5 antibody with a chelating agent (e.g., EDTA) has greater potency than a formulation without a chelating agent. For example, a formulation comprising an anti-C5 antibody (e.g., 10 mg/ml of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) is more potent than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)). Potency can be determined by any assay know in the art. Potency can also be determined by measuring the ability of the anti-C5 antibody to inhibit the activity of complement protein C5.

In one embodiment, a hemolysis assay is used determine the potency of an anti-C5 antibody. In one embodiment, the assay is a biological characterization method to quantify the inhibition of chicken erythrocyte lysis, an endpoint downstream of terminal complement activation, by an anti-C5 antibody. In this assay, varying concentrations of the anti-C5 antibody are incubated with a fixed concentration of normal human serum. This mixture is then incubated with chicken erythrocytes coated with rabbit anti-chicken erythrocyte antibodies. After incubation, the mixture is centrifuged, and the degree of hemolysis is quantified by measuring the absorbance (e.g., at A405 nm) of the hemoglobin released into the supernatant. The amount of complement activation correlates with the intensity of absorbance. Results can be reported as percent relative potency (% potency) values.

In another embodiment, potency is determined by a hemolytic assay for complement activation through the detection of a product resulting from terminal complement activation, in which the amount of product generated is proportional to the functional activity of complement. In one embodiment, the assay is an enzyme-linked immunosorbent assay (ELISA) that measures the ability of an anti-C5 antibody to inhibit the activation of complement protein C5 in human serum. The assay can use a labeled detection agent (e.g., specific an alkaline phosphatase labeled human C5b-9) monoclonal antibody) for a product (e.g., neo-antigen for which the human C5b-9 monoclonal antibody is specific for) produced as a result of terminal complement activation, in which the amount of product (e.g., C5b-9 neo-antigen) generated is proportional to the functional activity of complement. In one embodiment, varying concentrations of an anti-C5 antibody is incubated with a fixed concentration of normal human serum in the presence of a complement activator (e.g., zymosan). During incubation, normal human serum complement is activated by the zymosan and the C5b-9 complex that is generated as a result of terminal complement activation binds to the zymosan. C5b-9 is detected with an alkaline phosphatase labelled C5b-9 antibody and the amount of C5b-9 detected correlates with the amount of complement activation. The assay measures the anti-C5 antibody dose dependent detection of labelled C5b-9 (i.e., increasing antibody dose, decrease in detection of C5b-9 and thus complement activation). Test sample activity can be determined by comparing the test sample response to the response obtained with a reference standard (i.e., relative potency).

In one embodiment, a formulation comprising an anti-C5 antibody with a chelating agent (e.g., EDTA) has greater potency than a formulation without a chelating agent after a given time period (e.g., about 1 week, about 2 weeks; or about 1 day, about 2 days, about 3 days, about 4 days, about 5 days, about 6 days, about 7 days, about 8 days, about 9 days, about 10 days, about 11 days, about 12 days, about 13 days, about 14 days or about 15 days).

For example, a formulation comprising an anti-C5 antibody (e.g., 10 mg/ml of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) can be more potent than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)), after about 1 day, about 2 days, about 3 days, about 4 days, about 5 days, about 6 days, about 7 days, about 8 days, about 9 days, about 10 days, about 11 days, about 12 days, about 13 days, about 14 days or about 15 days.

In some embodiments, a formulation comprising an anti-C5 antibody with a chelating agent (e.g., EDTA) has reduced aggregation than a formulation without a chelating agent. For example, a formulation comprising an anti-C5 antibody (e.g., 10 mg/mL of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) has less aggregation than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)). Aggregation levels can be determined by methods known in the arts, such as by Size Exclusion Ultra High Performance Liquid Chromatography (SE-UHPLC) or Hydrophobic Interaction Chromatograph High Performance Liquid Chromatography (HIC-HPLC).

In one embodiment, a formulation comprising an anti-C5 antibody with a chelating agent (e.g., EDTA) has less aggregation than a formulation without a chelating agent after a given time period (e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks).

For example, a formulation comprising an anti-C5 antibody (e.g., 10 mg/ml of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) can have less aggregation than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)), after about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks.

In another example, a formulation comprising an anti-C5 antibody (e.g., 10 mg/mL of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) can have less aggregation than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)), after about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks; at about 50° C., 40° C., about 30° C., about 25° C., about 5° C., about −20° C. or about −30° C.

Without being bound by theory, an anti-C5 antibody (e.g., 10 mg/ml of eculizumab), a buffer (e.g., 10 mM acetate), a stabilizer (e.g., 5% sorbitol (w/v)), a surfactant (e.g., 0.01% polysorbate 80 (w/v)) and a chelating agent (e.g., 0.05 mM EDTA) at a particular pH (e.g., about 5.2) can have increased potency and/or less aggregation than the same formulation without a chelating agent such as EDTA (i.e., 10 mg/mL of eculizumab, 10 mM acetate, 5% sorbitol (w/v), 0.01% PS80, pH 5.2), or another formulation comprising a buffer, a tonicity agent, and a surfactant and no chelating agent (e.g., 10 mM sodium phosphate, 150 mM sodium chloride, 0.02% PS 80, pH 7.0)) because the anti-C5 antibody may have an oxidation modification in its heavy chain due to the presence of trace metals, which can lead to structural changes resulting in an increase in aggregate formation and/or a decrease of bioactivity. For example, eculizumab may have an oxidation modification on the heavy chain CDR3 (CDRH-3) tryptophan (position 9 of SEQ ID NO: 3, corresponding to position 107 of SEQ ID NO: 10), W107, which can lead to structural changes and aggregate formation, resulting in a decrease of bioactivity, due to the presence of trace metals. The presence of trace metals in a formulation with eculizumab may result in a higher percentage of HMW species and/or lower potency as compared to the formulations without detectable trace metals due to the oxidation of its CDRH-3. The instability of eculizumab in the formulation with detectable trace metals may be due to metal-catalyzed oxidation (e.g., iron-catalyzed oxidation, Fenton reaction) of eculizumab. The presence of a chelating agent in the formulation may counteract the effect of the presence of trace metals (e.g., inhibiting the iron-catalyzed oxidation, Fenton reaction), thus reducing the oxidation modifications of eculizumab, such as the oxidation of W107.

In some embodiments, eculizumab is manufactured in a single-use system (SUS). In some embodiments, eculizumab manufactured in a SUS has reduced oxidation modifications of eculizumab (e.g., oxidation of W107) as compared to eculizumab manufactured in a non-SUS. For example, in some embodiments, eculizumab manufactured in a SUS such that trace metals are reduced as compared to eculizumab manufactured in a non-SUS. In some embodiments, trace metals are not present or not detectable by conventional means, such as by inductively coupled plasma mass spectrometry (IPC-MS), in eculizumab drug product or drug substance manufactured in a SUS. Eculizumab manufactured with reduced trace metals and/or no detectable presence of trace metals may have increased stability and/or potency as compared to eculizumab with detectable trace metals (e.g., due to metal-catalyzed oxidation, such as iron-catalyzed oxidation or Fenton reaction of eculizumab, such as at the CRH-3 tryptophan (position 9 of SEQ ID NO: 3, corresponding to position 105 of SEQ ID NO: 10).

In some embodiments, eculizumab drug substance is manufactured in a SUS. In some embodiments, eculizumab drug product is manufactured in a SUS. In some embodiments, eculizumab drug substance and drug product are manufactured in a SUS. In some embodiments, a SUS comprises use of materials that are made from plastic materials and are disposable. For example, the container used in a SUS can be a commercially available single-use process container, such as those available from EMD Millipore (Burlington, MA), e.g., containers made with PureFlex™ film, such as a PureFlex™ bag with a product layer comprising of ultra-low density polyethylene (ULDPE), owhich can be used in the process for preparing or manufacturing drug product for the buffer, formulation, in-process hold and filling (e.g., surge vessel) unit operations. In some embodiments, the container used in a SUS are made of a material that does not have metals or does not leach metals. In some embodiments, the material is plastic. In some embodiments, the material is ethyl vinyl acetate (EVA).

In one embodiment, the level of metals detected in a drug substance (e.g., eculizumab drug substance) made using a SUS (e.g., such as the use of containers made of plastic rather than metal in one or more steps of the process) is lower than the drug substance made using a non-SUS (e.g., such as the use of stainless steel containers in one or more of the same corresponding step(s) of the process). In one embodiment, the SUS process comprises passing through viral filtered product through a SUS vessel or container (e.g., surge vessel) instead of a stainless steel vessel or container (e.g., surge vessel). In another embodiment, the SUS process comprises a SUS vessel or container (e.g., retentate vessel) for recovery of the product after UF/DF instead of a stainless steel vessel or container (e.g., retentate vessel). In some embodiments, the DS is stored in a SUS container (e.g., bag).

In another embodiment, the level of metals detected in a drug product (e.g., eculizumab drug product) made using a SUS (e.g., such as the use of containers made of plastic rather than metal in one or more steps of the process) is lower than the drug product made using a non-SUS (e.g., such as the use of stainless steel containers in one or more of the same corresponding step(s) of the process). The formulation and/or hold tank(s) where the drug product is held, such as for a period of time, can be made from these plastic materials (e.g., EVA) in a SUS. The plastic material can allow the drug product to have minimal to no metal contact, such that no metal will be leached. In one embodiment, the level of metals is minimal, such as undetectable, in a drug product (e.g., eculizumab) made using SUS. Non-SUS primarily comprises the use of stainless steel (SS) components instead of single-use containers. For example, the formulation and/or hold tank(s) where the drug product is held for a period of time is made from SS rather than a single-use container, such as a container made from plastic materials. In some embodiments, these SS components have the potential to leach metals into the formulated or filtered drug product, which may increase the degradation of the protein.

The detailed description and following examples illustrate the present invention and are not to be construed as limiting the present invention thereto. Various changes and modifications can be made by those skilled in the art on the basis of the description of the invention, and such changes and modifications are also included in the present invention.

### EXAMPLE

### Example 1

Development of a formulation with freeze-thaw stability and ability to be stored at different temperature conditions was performed. The liquid stability of eculizumab (SEQ ID NO: 10) at a concentration of 10 mg/ml in a sorbitol formulation was evaluated by size-exclusion ultrahigh-performance liquid chromatography (SE-UHPLC), peptide mapping, hydrophobic interaction chromatography-high performance liquid chromatography (HIC-HPLC), and an ELISA-based potency assay.

Eculizumab at a concentration of 10 mg/mL in a sorbitol formulation ((10 mM acetate, 5% sorbitol (w/v), 0.01% polysorbate 80 (w/v), pH 5.2) showed product instability as compared to eculizumab at a concentration of 10 mg/ml in a PBS formulation (10 mM sodium phosphate, 150 mM sodium chloride, 0.02% polysorbate 80 (w/v), pH 7.0) based on potency data generated from an ELISA-based potency assay, both at the storage condition of 5° C. and under accelerated conditions of 40° C. and forced degradation conditions of 50° C.

In this ELISA-based potency assy, the ability of eculizumab to inhibit the activation of complement protein C5 in human serum was measured through determining the amount of C5b-9 neo-antigen generated, which is proportional to the functional activity of complement. Varying concentrations of eculizumab were incubated with a fixed concentration of normal human serum (NHS) in the presence of zymosan (a complement activator). During incubation, normal human serum complement was activated by the zymosan coating on the assay plates and the C5b-9 complex that was generated as a result of terminal complement activation binding to the zymosan. The plate wells were then washed and C5b-9 detected with an alkaline phosphatase labelled C5b-9 antibody. After addition of an alkaline phosphatase substrate solution to the plate wells, absorbance was measured at 405 nm. The amount of complement activation correlates with the absorbance at 405 nm, which provides the measurement of the eculizumab dose dependent decrease at 405 nm absorbance. The activity was then determined by comparing the sample response to the response obtained with the reference standard (Relative Potency).

It was thought that the instability may be caused by low levels of trace metals (<1 ppm), as spiking of iron into a PBS formulation with eculizumab resulted in the same degradation profile as determined by HIC-HPLC and peptide mapping analysis that was observed with eculizumab in the sorbitol formulation.

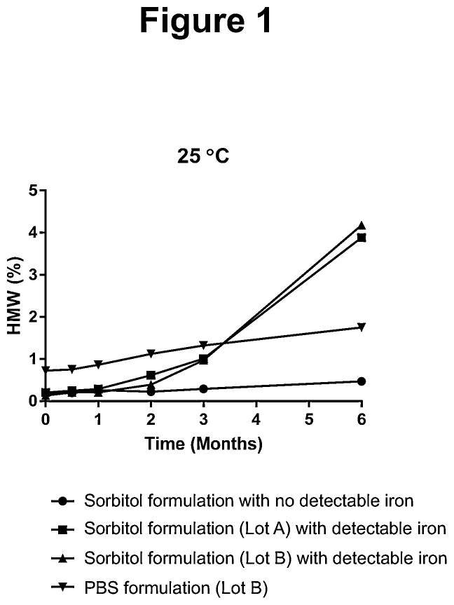

To determine if this was the case, 10 mg/ml of eculizumab in a sorbitol formulation (10 mM acetate, 5% sorbitol (w/v), 0.01% polysorbate 80 (w/v), pH 5.2) with no detectable trace metals, e.g., iron (detected by IPC-MS), 10 mg/ml of eculizumab in a sorbitol formulation (10 mM acetate, 5% sorbitol (w/v), 0.01% polysorbate 80 (w/v), pH 5.2) with detectable trace metals (two different lots of eculizumab were formulated in the sorbitol formulation, Lot A and Lot B) and 10 mg/ml of eculizumab in a PBS formulation (10 mM sodium phosphate, 150 mM sodium chloride, 0.02% polysorbate 80 (w/v), pH 7.0) were stored at 25° C. for six months and analyzed by SE-UHPLC at 2 weeks, 1 month, 2 months, 3 months and 6 months (FIG. 1). SE-UHPLC separates proteins based on differences in their hydrodynamic volumes, in which molecules with larger hydrodynamic volumes elute earlier than molecules with smaller volumes. The samples were loaded onto an SE-UHPLC column (BEH200, UPLC column, 4.6 mm×150 mm, 1.7 μm (Waters Corp., 186005225), separated isocratically with sodium phosphate/sodium chloride buffer, and the eluent monitored by UV absorbance (280 nm). Purity was determined by calculating the percentage of each separated component as compared to the total integrated area (the levels of HMW aggregates were calculated by determining the total area of HMW peaks over the total peak area).

The percentage of high molecular weight (HMW) species as detected by SEC-UHPLC increased at a faster rate in the sorbitol formulation with detectable trace metals as compared to the sorbitol formulation with no detectable trace metals, as well as compared to the PBS formulation.

As the sorbitol formulation with detectable trace metals (e.g., iron) resulted in a higher percentage of HMW species as compared to the sorbitol formulation without detectable trace metals, it was thought that instability of eculizumab in the sorbitol formulation with detectable trace metals may be due to metal-catalyzed oxidation (e.g., iron-catalyzed oxidation, Fenton reaction) of eculizumab. Peptide mapping of eculizumab in the sorbitol formulation was performed. The sample was reduced, and then excess reagents are removed by size exclusion-based desalting columns before digestion with trypsin or Asp-N. The resulting peptides are then separated by RP-HPLC in a trifluoroacetic acid/acetonitrial (TFA/ACN) gradient and monitored by UV at 214 nm with MS and MS/MS data collection. The levels of each type of post-translational modification (PTM) was compared to the integrated peak area in the UV trace of the modified peptide containing the residue of interest with that of the peptide containing both the unmodified and modified residues, which is obtained by using Mass Analyzer Software.

Results showed the formation of +16 Da, +32 Da and +14 Da peptides, representing oxidation modifications on the heavy chain CDR3 (CDRH-3) tryptophan (position 9 of SEQ ID NO: 3, corresponding to position 107 of SEQ ID NO: 10), W107. These modifications are likely followed by structural changes and aggregate formation, resulting in a decrease of bioactivity. The relative potency of eculizumab in a sorbitol formulation with detectable trace metals was determined to be lower than eculizumab in a sorbitol formulation with no detectable trace metals, and this loss in potency was determined to be due to the oxidation of W107 as determined by reduced peptide mapping.

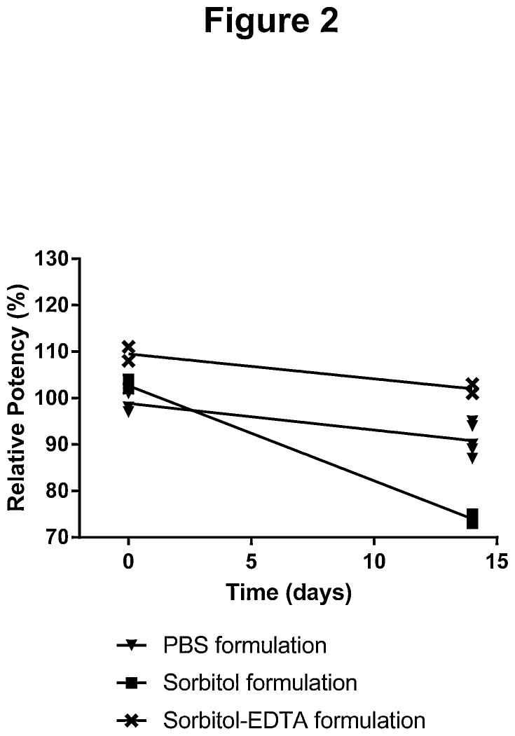

To determine if the decrease in potency of eculizumab in the sorbitol formulation with detectable trace metals could be decreased (e.g., by inhibiting the metal-catalyzed oxidation of eculizumab), EDTA was added to the sorbitol formulation. The relative potency of the eculizumab (10 mg/ml) in the sorbitol formulation with EDTA (10 mM acetate, 5% sorbitol (w/v), 0.01% polysorbate 80 (w/v), 0.05 mM EDTA, pH 5.2) was compared to eculizumab (10 mg/ml) in the PBS formulation (10 mM sodium phosphate, 150 mM sodium chloride, 0.02% polysorbate 80 (w/v), pH 7.0) and sorbitol formulation without EDTA (10 mM acetate, 5% sorbitol, 0.01% polysorbate 80, pH 5.2) at an accelerated condition of 50° C. for two weeks (FIG. 2). The potency of eculizumab in the sorbitol formulation decreased at a much greater rate as compared to eculizumab in the sorbitol formulation with EDTA and eculizumab in the PBS formulation.

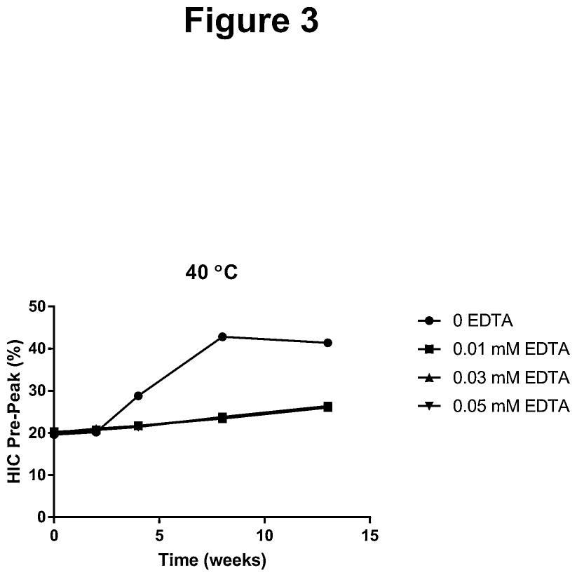

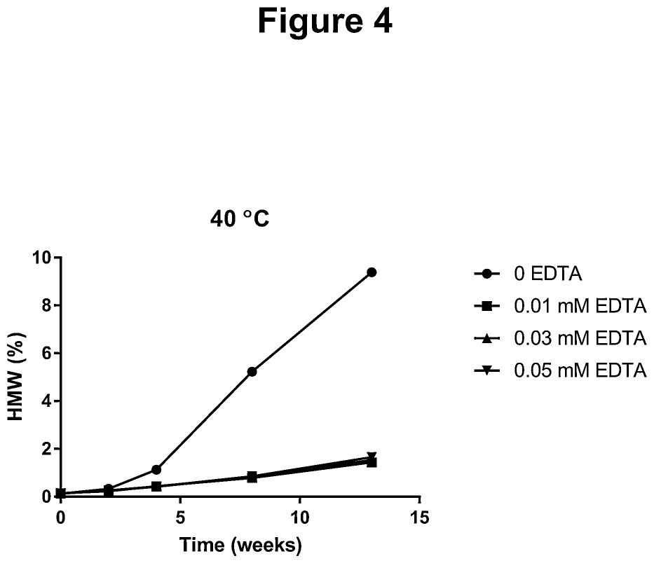

To determine if the amount of EDTA in a formulation can control the level of oxidation of eculizumab, varying amounts of EDTA (0, 0.01 mM, 0.03 mM, and 0.05 mM) were included in the sorbitol formulation (10 mM acetate, 5% sorbitol (w/v), 0.01% polysorbate 80 (w/v), pH 5.2) in which eculizumab was present at 10 mg/ml. The formulations were stored at 40° C. for 13 weeks. The formation of oxidized forms of eculizumab was measured by HIC-HPLC Prepeaks (FIG. 3). HIC-HPLC was used for quantitative purity analysis of ABP 959 tryptophan oxidized species. Samples are loaded onto two HIC-HPLC columns (Propac HIC-10, 5 μm, 4.6×100 mm (Thermo, 063655)) connected in series, separated in a decreasing salt gradient of ammonium sulfate and sodium acetate buffer, and the eluent monitored by ultraviolet (UV) absorbance at 220 nm. Purity was determined by calculating the percentage of each separated component as compared to the total integrated area. The level of oxidized tryptophan has a linear correlation correlation with the level of percentage pre-peaks determined by HIC-HPLC analysis (the percentage of pre-peaks is equal to the pre-peaks area over the total integrated peak area). The level of aggregation (as measured by percentage of HMW species) was determined by SEC-UHPLC (FIG. 4). As shown in FIGS. 3 and 4, respectively, the highest amount of oxidation and HMW species was found in the formulation without EDTA.

This example demonstrates a sorbitol formulation with EDTA for eculizumab that provides superior protein solubility and stability by preventing metal catalyzed oxidation (via the Fenton Reaction) and subsequent aggregation, precipitation and other chemical modifications was developed. This formulation of eculizumab with EDTA offers superior stability not only over a PBS formulation, but also a sorbitol formulation without EDTA.

### Example 2

A comparison of eculizumab drug product (DP) produced using a non-single use system (SUS) to eculizumab DP using a SUS was performed.

The same drug substance process was performed for both SUS and non-SUS DP. The cell culture process was performed in shake flasks, Wave Bioreactor™ (Cytvia, Marlborough, MA, USA), 500 L single use bioreactor (SUB), and 2000 L production SUB. The cells were harvested using alternating tangential flow (ATF) filters and held in a SUS (polyethylene (PE) film, Thermo Scientific™ ASI™ imPULSE Single Use Mixer) before being processed for purification with Protein A. The elutate from Protein A purification was titrated to low pH for viral inactivation and neutralization, then filtered and processed over a cation exchange column (CEX). The eluate from the CEX was then processed with Mixed Mode Anion Exchange Chromatography (MMA). The MMA flow-through was then processed through viral filtration. The viral filtered pool underwent ultrafiltration/diafiltration (UF/DF) in a connected process, which allowed the viral filtered pool to be buffer exchanged into the formulation buffer and concentrated to the target concentration for eculizumab drug substance (DS). The recovered UF/DF pool was then spiked with polysorbate 80 (PS80) and filtered into SUS bags and stored at −30° C.

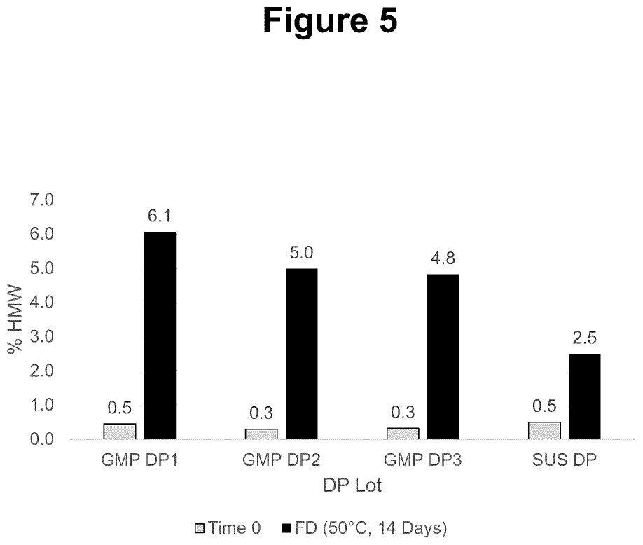

In the non-SUS DP, the DS is formulated at a concentration of 10 mg/ml in a sorbitol formulation (10 mM acetate, 5% sorbitol, 0.01% polysorbate 80, pH 5.2) in a non-SUS formulation tank (stainless steel, SS) before being filtered through a 0.22 μm PVDF filter, then held in a non-SUS (SS) hold tank, filtered again with a 0.22 μm PVDF filter, then held in a surge SUS bag before filling. The eculizumab DP produced using this method were designated GMP lots (see FIGS. 5-8).

In the SUS for DP, the process was the same as the non-SUS DP process except for the use of SUS ethyl vinyl acetate (EVA) bags instead of SS tanks (i.e., instead of a SS formulation tank, a SUS EVA bag was used; instead of a SS hold tank, a SUS EVA bag was used). The eculizumab DP produced using this method were designated as SUS lot (see FIGS. 5-8).

The percentage of high molecule weight (HMW) species, determined by SE-UHPLC method (as descried in Example 1), in eculizumab DP lots produced using the non-SUS (GMP DPI, GMP DP2, and GMP DP3) were compared to the percentage of HMW in an eculizumab DP lot produced using SUS (SUS DP) under forced degradation conditions of 50°0 C. for 14 days. The percentage of HMW species or aggregates were measured and is shown in FIG. 5. As FIG. 5 shows, the percentage of HMW in the SUS eculizumab DP lot under forced degradation conditions was greatly reduced as compared to the non-SUS eculizumab DP lots under forced degradation conditions.

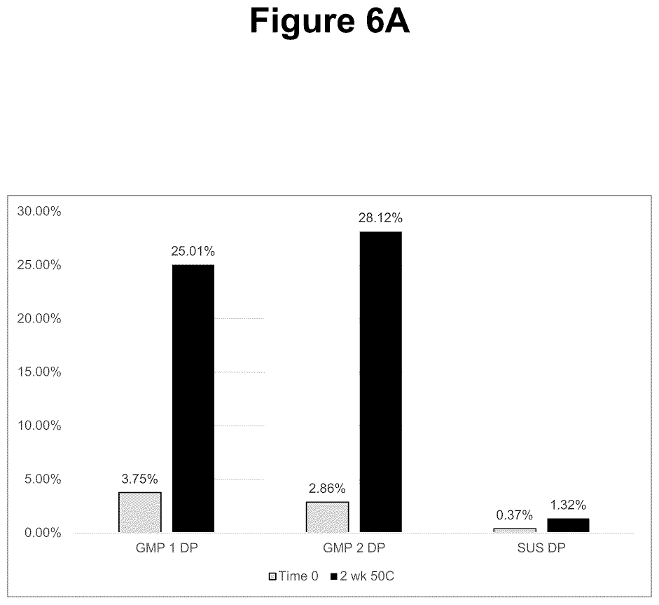

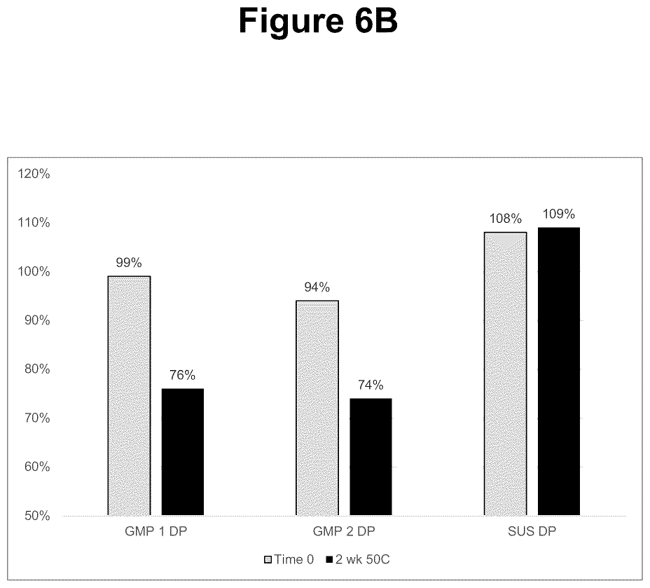

The amount of oxidation modification on the heavy chain CDR3 (CDRH-3) tryptophan (position 9 of SEQ ID NO: 3, corresponding to position 107 of SEQ ID NO: 10), W107, in the eculizumab DP lots produced using the non-SUS (GMP 1 DP, GMP 2 DP) were compared to the eculizumab DP lot produced using SUS (SUS DP) under forced degradation conditions was also determined by peptide mapping (as described in Example 1). FIG. 6A shows that the level of W107 oxidation is much lower for the SUS eculizumab DP lot under forced degradation conditions as compared to the levels in non-SUS eculizumab DP lots (GMP 1 DP, GMP 2 DP) under forced degradation conditions.

The potency of the eculizumab DP lots produced using the non-SUS (GMP 1 DP, GMP 2 DP) under forced degradation conditions were compared to the eculizumab DP lot produced using the SUS (SUS DP) under forced degradation conditions using the ELISA-based potency assay described in Example 1. As shown in FIG. 6B, no loss of potency was seen in the SUS eculizumab DP lot, in contrast to the non-SUS eculizumab DP lots.

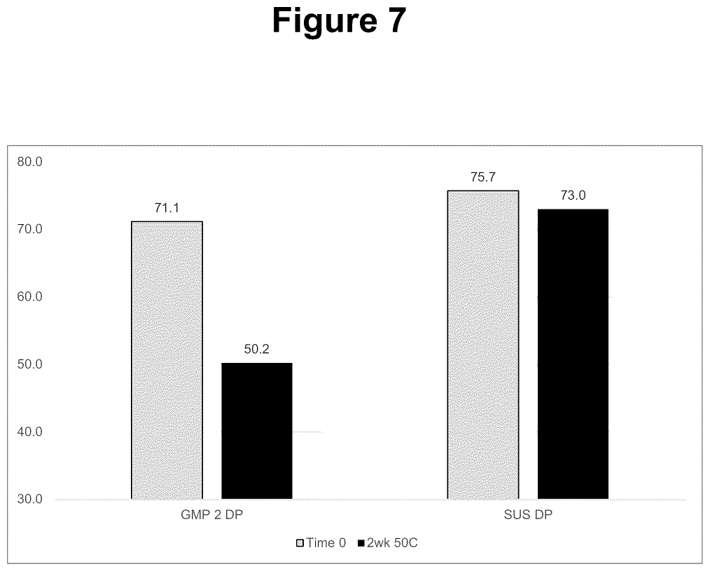

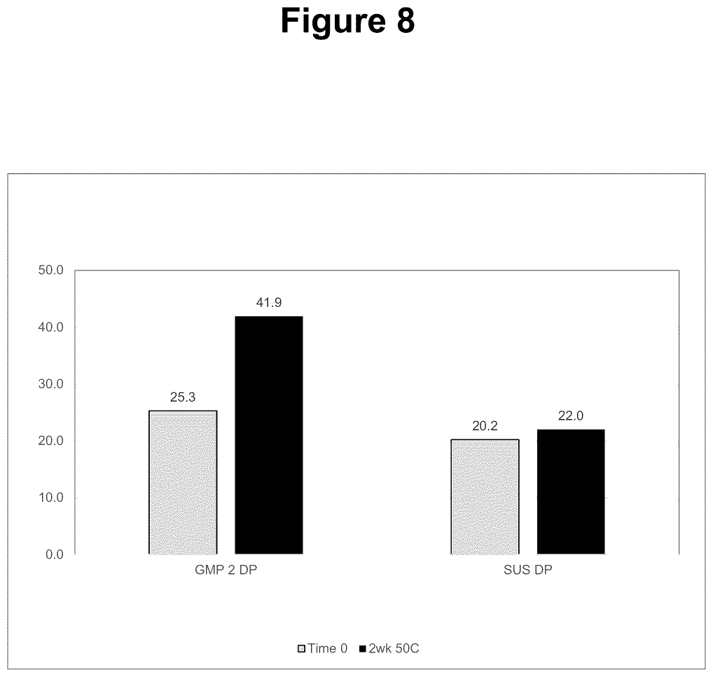

In addition, HIC-HPLC analysis (as performed in Example 1) showed that the SUS eculizumab DP lot under forced degradation conditions did not exhibit loss in HIC Main Peak as compared to the non-SUS eculizumab DP lot (GMP 2 DP) under forced degradation conditions, as shown in FIG. 7, and the SUS eculizumab DP lot under forced degradation conditions did not show a loss of purity, as demonstrated by the percentage of HIC Pre-Peaks (FIG. 8).

While the present invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described. All references cited in this application are expressly incorporated by reference herein for any purpose.

1. A formulation comprising:
a) an anti-C5 antibody, wherein the antibody comprises a CDRH1, CDRH2, and CDRH3 having the amino acid sequence of SEQ ID NOs: 1, 2 and 3, or SEQ ID NOs: 4, 5, and 3, respectively; and a CDRL1, CDRL2, and CDRL3 having the amino acid sequence of SEQ ID NO: 12, 13 and 14, respectively;
b) a buffer comprising acetate at a concentration of between 5 mM and 20 mM,
c) a stabilizer, wherein the stabilizer is a polyol, and
d) a chelating agent, wherein the concentration of the chelating agent is between 0.01 mM and 0.05 mM.

2.-4. (canceled)

5. The formulation of claim 1, wherein the antibody comprises a heavy chain having the amino acid sequence of SEQ ID NO: 10 or 11.

6.-8. (canceled)

9. The formulation of claim 1, wherein the antibody comprises a light chain having the amino acid sequence of SEQ ID NO: 17.

10.-14. (canceled)

15. The formulation of claim 1, wherein the concentration of the acetate is about 10 mM.

16. (canceled)

17. The formulation of claim 1, wherein the polyol is sorbitol.

18. The formulation of claim 17, wherein the concentration of sorbitol is about 5% (w/v).

19. (canceled)

20. The formulation of claim 1, wherein the concentration of the chelating agent is about 0.05 mM.

21. The formulation of claim 1, wherein the chelating agent is ethylenediaminetetraacetic acid (EDTA).

22. The formulation of claim 1, further comprising a surfactant, wherein the concentration of the surfactant is between 0.001% and 0.1% (w/v).

23. (canceled)

24. The formulation of claim 22, wherein the surfactant is polysorbate 80.

25. The formulation of claim 1, wherein the pH of the formulation is between 4.5 and 5.8.

26. (canceled)

27. The formulation of claim 1, wherein the concentration of the antibody is about 10 mg/ml.

28. (canceled)

29. A method of manufacturing an anti-C5 antibody drug product comprising:
a. formulating an anti-C5 antibody drug substance in a formulation comprising a buffer, a stabilizer, and optionally, a chelating agent, in a single use system formulation container;
b. filtering the resulting drug product a first time;
c. holding the drug product in a single use system hold container;
d. filtering the drug product a second time; and
e. holding the drug product in a surge container,
wherein the drug product has fewer aggregates, lower oxidation levels, decreased loss of potency, or decreased loss of purity, as compared to a drug product manufactured with the same process except for the use of a formulation container, a hold container, or both a formulation and hold container, comprising metal.

30. The method of claim 29, wherein the formulation container, the hold container or both the formulation and hold container, does not contain metal.

31.-60. (canceled)

61. The method of claim 30, wherein the formulation comprises:
a) about 10 mM acetate;
b) about 5% (w/v) sorbitol; and
c) about 0.05 mM ethylenediaminetetraacetic acid (EDTA).

62. The method of claim 61, wherein the formulation further comprises 0.01% (w/v) polysorbate 80.

63. A formulation comprising:
a) about 10 mg/ml of an anti-C5 antibody comprising a heavy chain having the amino acid sequence of SEQ ID NO: 10 and a light chain having the amino acid sequence of SEQ ID NO: 17;
b) about 10 mM acetate;
c) about 5% (w/v) sorbitol;
d) about 0.05 mM ethylenediaminetetraacetic acid (EDTA),
wherein the formulation has a pH of about 5.2.

64. A formulation comprising:
a) about 10 mg/ml of an anti-C5 antibody comprising a heavy chain having the amino acid sequence of SEQ ID NO: 11 and a light chain having the amino acid sequence of SEQ ID NO: 17;
b) about 10 mM acetate;
c) about 5% (w/v) sorbitol;
d) about 0.05 mM ethylenediaminetetraacetic acid (EDTA),
wherein the formulation has a pH of about 5.2.

65. The formulation of claim 63 further comprising 0.01% (w/v) polysorbate 80.

66. The formulation of claim 64 further comprising 0.01% (w/v) polysorbate 80.


SEQUENCE LISTING (17 sequences)

SEQ ID NO: 1 - protein, 10 aa, Artificial Sequence
  DOE Sequence
GYIFSNYWIQ

SEQ ID NO: 2 - protein, 17 aa, Artificial Sequence
  DOE Sequence
EILPGSGSTEYTENFKD

SEQ ID NO: 3 - protein, 13 aa, Artificial Sequence
  DOE Sequence
YFFGSSPNWYFDV

SEQ ID NO: 4 - protein, 10 aa, Artificial Sequence
  DOE Sequence
GHIFSNYWIQ

SEQ ID NO: 5 - protein, 17 aa, Artificial Sequence
  DOE Sequence
EILPGSGHTEYTENFKD

SEQ ID NO: 6 - protein, 122 aa, Artificial Sequence [sequence omitted]

SEQ ID NO: 7 - protein, 122 aa, Artificial Sequence [sequence omitted]

SEQ ID NO: 8 - protein, 326 aa, Artificial Sequence
  DOE Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 326 aa, Artificial Sequence
  DOE Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 10 - protein, 448 aa, Artificial Sequence
  DOE Sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 448 aa, Artificial Sequence
  DOE Sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 12 - protein, 11 aa, Artificial Sequence
  DOE Sequence
GASENIYGALN

SEQ ID NO: 13 - protein, 7 aa, Artificial Sequence
  DOE Sequence
GATNLAD

SEQ ID NO: 14 - protein, 9 aa, Artificial Sequence
  DOE Sequence
QNVLNTPLT

SEQ ID NO: 15 - protein, 107 aa, Artificial Sequence [sequence omitted]

SEQ ID NO: 16 - protein, 107 aa, Artificial Sequence [sequence omitted]

SEQ ID NO: 17 - protein, 214 aa, Artificial Sequence [sequence omitted]